(19) United States
(12) Patent Application Publication
(10) Pub. No.: US 2026/0053537 A1
(43) Pub. Date: Feb. 26, 2026

(54) OSTEOSYNTHESIS IMPLANT SYSTEM

(71) Applicant: Karl Leibinger Asset Management GmbH & Co. KG, Mühlheim an der Donau (DE)

(72) Inventors: Ralf Leibinger, Mühlheim (DE); Artur Kühn, Radolfzell (DE); Frank Reinauer, Emmingen-Liptingen (DE)

(21) Appl. No.: 19/307,615

(22) Filed: Aug. 22, 2025

(30) Foreign Application Priority Data
Aug. 26, 2024 (DE) 102024124324.4

(51) Int. Cl. A61B 17/80; A61B 17/86

(52) U.S. Cl. CPC A61B 17/8057; A61B 17/8071; A61B 17/8605

(57) ABSTRACT

An osteosynthesis implant system (1; 1a) includes a bone screw (K1; K2, K3; K1a, K1b) having a screw head (SK1) and a screw shank and also includes an osteosynthesis plate (10; 10′; 10a) with at least one slot (L1, L2; L1′, L2; L1a-L6a) through which the bone screw (K1; K2, K3; K1a, K1b) is bringable into engagement at different positions to be anchored to the bone (SK, OK). The slot (L1, L2; L1′, L2; L1a-L6a) includes a first and a second longitudinal bar (LL1, LL2) that extend along an extension direction of the osteosynthesis plate (10; 10′; 10a) and have a bevel (F1) which is directed into the slot (L1, L2; L1′, L2; L1a-L6a). T thread projection (S1) for accommodating a thread turn (GK1) of the screw shank is formed on the first longitudinal bar (LL1) underneath the bevel (F1). A flat surface (EF) is formed on the second longitudinal bar (LL2) underneath the bevel (F1) and extends substantially in parallel with a depth extent (T) of the slot (L1, L2; L1′, L2; L1a-L6a).

### CROSS-REFERENCE TO RELATED APPLICATION

The present application is related and has right of priority to German Patent Application No. DE102024124324.4 filed on Aug. 26, 2024, which is incorporated by reference in its entirety for all purposes.

### TECHNICAL FIELD

The present invention relates generally to an osteosynthesis implant system.

### BACKGROUND

Fractures of bones on the endoskeleton of mammals result in protracted healing processes. An osteosynthesis implant system is used to promote an osteosynthetic process at a gap at a bone. The fractures are stabilized here by attaching osteosynthesis plates to the respective bones using fasteners, for example, screws, wherein sections of the bones are fixed in relation to one another.

U.S. Pat. No. 4,696,290 A describes an osteosynthesis plate for connecting vertebrae, which has an elongated slot for accommodating a screw, wherein the screw can be inserted at any position.

U.S. Pat. No. 8,852,249 B2 discloses an osteosynthesis plate having a bottom side that is to rest against the bone, a top side facing away from the bone, and a plurality of holes along the longitudinal axis of the plate, through which bone screws can be inserted to be anchored to the bone, wherein at least one hole is a continuous slot having the longitudinal axis extending in the direction of the longitudinal axis of the plate, wherein thread flights are provided in a portion of the lateral edge of the slot that, as viewed in a direction transversal to the plane of the top side, are arranged only over a part of the depth of the slot. As viewed in the direction transversal to the plane of the top side, a retaining structure having smooth walls is provided for the positive fit with a correspondingly configured negative structure at a screw head or a screw neck of a bone screw.

U.S. Pat. No. 2019/0216611 A1 describes an osteosynthesis plate having at least one slot, which has grooves on the longitudinally extending, facing walls for accommodating a bone screw.

In the implant treatment in the course of an osteosynthesis, the problem often arises that positions of bone screws can not be reached or can be only partially reached. The reasons are many, for example, a small wound opening that is gentle on the patient, or anatomy or instruments for the surgical procedure located in the immediate vicinity.

Access is frequently limited, which prevents the screwdriver from being properly positioned when tightening the screw.

Known implants used in osteosynthesis frequently have defined or very narrowly defined bone screw positions with defined orientations. Freely selectable positions are either not available or are available only with limitations, for example, without self-retention of the screw connection.

### BRIEF SUMMARY

Example aspects of the present invention provide an improved osteosynthesis implant system, which is simply designed and offers increased flexibility for the positions of the bone screws.

Example aspects of the invention provide, in addition to the defined and easily reachable positions of the bone screws, further options for freely inserting and orienting the bone screws.

Specifically, example aspects of the invention relate to an osteosynthesis implant system having at least one bone screw and one osteosynthesis plate. The bone screw has a screw head and a screw shank. The osteosynthesis plate has a bottom side that is to rest against the bone and a top side facing away from the bone. The osteosynthesis plate also has at least one slot through which the bone screw is bringable into engagement at different positions to be anchored to the bone. The slot has a first and a second longitudinal bar which extend along an extension direction of the osteosynthesis plate and are oriented in parallel with one another at least partially or in sections. The longitudinal bars have, on the top side, a bevel directed into the slot.

The bevel is configured such that the screw head, when fastened on the bone, is at least partially accommodated in the slot, such that a contact surface for the screw head is provided on the surface formed by the bevel.

The first longitudinal bar has a thread projection underneath the bevel, i.e., in the direction of the bottom side proceeding from the bevel. The thread projection is designed to accommodate a thread turn of the screw shaft. The second longitudinal bar has a flat surface underneath the bevel, which flat surface extends substantially in parallel with a depth extent of the slot. Due to the thread projection being formed on one side, the bone screw can also contribute to the fixation of the osteosynthesis plate on the bone when the bone screw is tightened obliquely to the orientation of the slot. In such a case, the screw head comes to rest on the surface of the bevel in an undefined manner. The screw holds in this case due to the tension between the thread turn on the screw shaft and the thread projection arranged on one side. Since the thread projection is arranged on only one side, it is much easier for the surgeon to insert the screw into the slot than it would be in a design in which the thread projection is formed on both longitudinal bars. The design in which a thread projection is formed on only one side also allows for a greater variance in possible angular positions in which the screw can be tightened.

Preferably, the second longitudinal bar does not have a geometry which is designed to accommodate a thread turn or the thread turn of the bone screw. In other words, the flat surface is free of projections, bars, or notches which could be used to accommodate a thread turn.

Preferably, the flat surface extends between the top side and the bottom side.

Moreover, both the bevel on the top side and a bevel that is arranged on the bottom side if necessary are to be taken into account, such that the flat surface extends between these bevels.

According to a preferred example development, the slot is annularly closed, wherein the bevel extends peripherally on the top side of the slot.

According to a further preferred example development, the bone screw is bringable into engagement in the slot at the different positions with a variable inclination with respect to the depth extent of the slot to be anchored to the bone. This provides increased flexibility when fastening the osteosynthesis plate on the bone. For example, the inclination can be approximately ten to twelve degrees (10-12°) in any direction, so that, overall, an angle range of approximately twenty to twenty-four degrees (20-24°) is available.

According to a further preferred example development, the screw head of the bone screw is lenticular. A lenticular form is understood to mean that the screw head in cross-section has an elliptical shape similar to a convex lens. Due to such a shape, a good retention of the screw head in the bevel surface of the slot is made possible when the bone screw is inserted straight into the slot, i.e., in the direction of the depth extent of the slot. If the screw is inserted obliquely into the slot, however, this shape allows for a good remaining surface for the placement of the screw head in the bevel surface, particularly in comparison to a flat contact surface of the screw head oriented orthogonally to the axis of the screw.

According to a further preferred example development, the thread projection is formed by a single, preferably polygonal or rounded, projection. In other words, in such an example embodiment, only one single projection is present, in which precisely one thread turn of the bone screw is accommodated. Polygonal is understood to mean a geometry in which the thread projection is formed by multiple angled sections. By comparison, a rounded projection is formed by round shapes, wherein the radius of the round shapes can vary.

According to a further preferred example development, the thread projection is formed by at least two consecutively arranged individual projections. This brings about a further improvement of the retention force, since multiple thread projections engage into the thread turn of the bone screw.

Preferably, the thread projection is arranged at half the height between a lower edge of the bevel and the edge of the first longitudinal bar to the bottom side of the osteosynthesis plate. Such a design has proven advantageous for the simple insertion of the screw.

Preferably, the thread projection has a non-symmetrical profile. A design has proven particularly advantageous, in which the slope of the side of the thread projection facing away from the bone is greater than a slope of the side of the thread projection facing the bone. Due to such a design, a particularly good retention of the screw connection is ensured.

According to a further preferred example development, the osteosynthesis plate has at least one round hole through which an additional bone screw is bringable into engagement at a fixedly defined position to be anchored to the bone.

According to a further preferred example development, a first plurality of slots and a second plurality of round holes are provided, at least one of the round holes adjoining one of the slots in the longitudinal direction.

According to a further preferred example development, the slot has a first section and a second section in which the longitudinal bars extend in parallel in each case, and wherein the first section and the second section extend at an angle relative to one another. A good anatomical adaptation can be achieved in this way.

According to a further preferred example development, one or more marker(s) is/are provided along the longitudinal bars on the top side, which indicate target positions of the bone screw in the slot. This facilitates the positioning of the bone screws for the surgeon.

Preferably, the osteosynthesis plate forms a maxilla repositioning implant for orienting a maxilla displacing portion relative to a remaining maxilla portion fixed to the skull. The approach according to example aspects of the invention is particularly well suited for such an implant, since a wound opening in the facial region is to be kept as small as possible, and therefore the screw fixing positions are reachable only in a limited manner in some cases.

### BRIEF DESCRIPTION OF THE DRAWINGS

Exemplary embodiments of the invention are explained in the following with reference to the accompanying figures. Wherein:

FIGS. 1a-1d show schematic views of an osteosynthesis implant system according to a first example embodiment, specifically FIG. 1a showing a perspective view of a connection state between a skull bone and an upper jaw bone, FIG. 1b showing a perspective view, in sections, of a bone screw inserted in a slot, FIG. 1c showing a cross-sectional view, in sections, of the bone screw inserted in the slot, and FIG. 1d showing a further perspective view, in sections, of a bone screw inserted in a modified slot;

FIGS. 2a-2b show schematic views of an osteosynthesis implant system according to a second example embodiment, specifically FIG. 2a showing a perspective view of a planned connection state between a skull bone and an upper jaw bone, and FIG. 2b showing a perspective view of a connection state between a skull bone and an upper jaw bone with two bone screws inserted in slots; and

FIGS. 3A-3C show schematic sectional views of example variants of the thread projection.

In the figures, identical or functionally identical elements and devices have been provided with the same reference characters, unless otherwise stated.

### DETAILED DESCRIPTION

Reference will now be made to embodiments of the invention, one or more examples of which are shown in the drawings. Each embodiment is provided by way of explanation of the invention, and not as a limitation of the invention. For example, features illustrated or described as part of one embodiment can be combined with another embodiment to yield still another embodiment. It is intended that the present invention include these and other modifications and variations to the embodiments described herein.

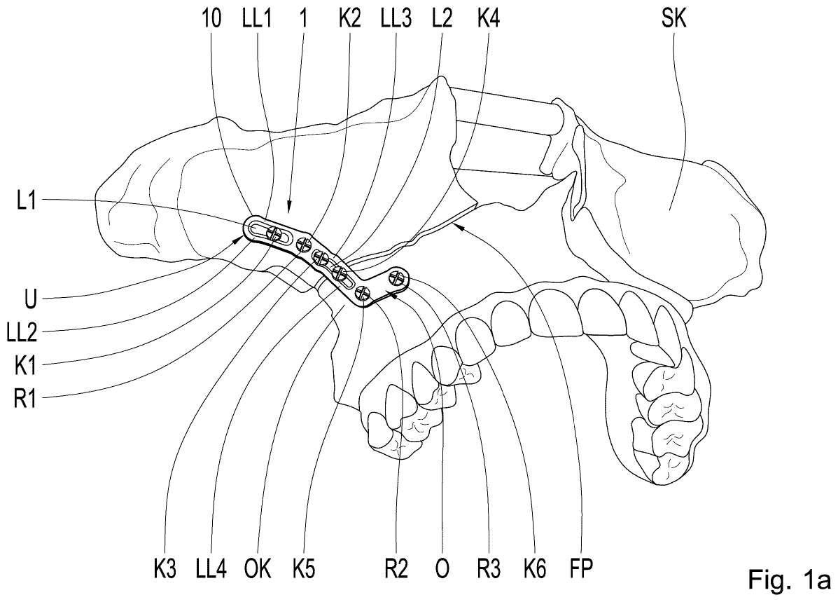

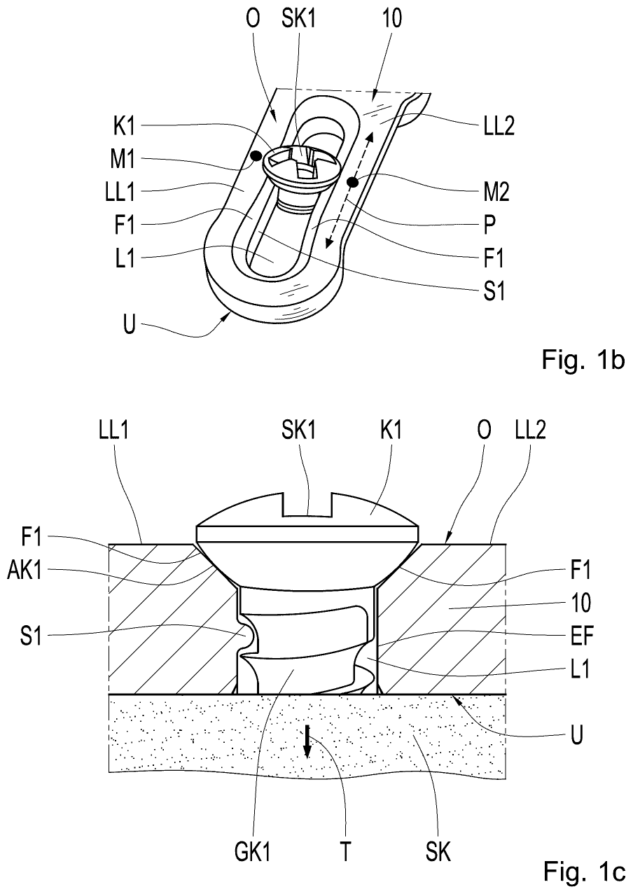

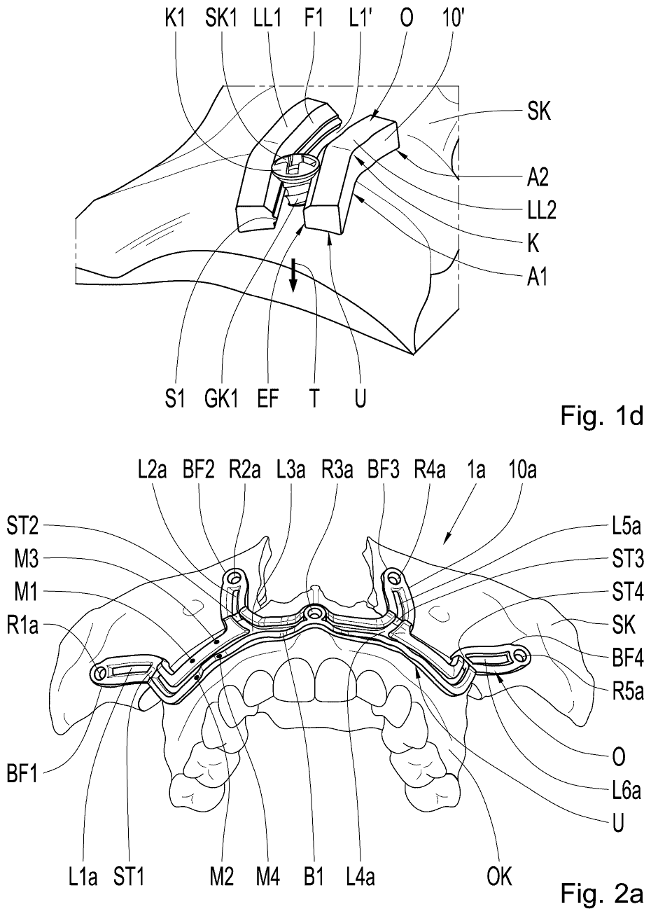

FIG. 1a through FIG. 1d are schematic views of an osteosynthesis implant system according to a first example embodiment, specifically FIG. 1a showing a perspective view of a connection state between a skull bone and an upper jaw bone, FIG. 1b showing a perspective view, in sections, of the bone screw inserted in a slot, FIG. 1c showing a cross-sectional view, in sections, of the bone screw inserted in the slot, and FIG. 1d showing a further perspective view, in sections, of a bone screw inserted in a modified slot.

With reference to FIG. 1a, reference character 1 generally designates the osteosynthesis implant system according to the first example embodiment, which is shown in an implanted state in which it is used to stabilize a gap FP between an upper jaw bone OK and a skull bone SK. The gap FP can be present due to a fracture, or due to an osteotomy and subsequent displacement of the upper jaw bone OK relative to the skull bone SK.

The osteosynthesis implant system 1 includes bone screws K1 through K6 and an osteosynthesis plate 10 having a bottom side U that is to rest against the bone and a top side O facing away from the bone.

The osteosynthesis plate 10 has a first and a second slot L1, L2. The bone screw K1 is bringable into engagement in the first slot L1 at different positions to be anchored to the skull bone SK (see arrow P in FIG. 1b), and the bone screws K3, K4 are bringable into engagement in the second slot L2 at different positions to be anchored to the skull bone SK and to the upper jaw bone OK, respectively. The slot L1 has two parallel longitudinal bars LL1, LL2, and the slot L2 has two parallel longitudinal bars LL3, LL4.

The slots L1, L2 extend along an extension direction of the osteosynthesis plate 10.

The osteosynthesis plate 10 also has a first through third round hole R1 through R3 in which the bone screws K2, K5, K6 are bringable into engagement at a fixedly defined position to be anchored to the skull bone SK and to the upper jaw bone OK.

Furthermore, with reference to FIGS. 1b and 1c, the slot L1 has, on the top side O, a bevel F1, which is directed into the slot L1 and is configured such that a lenticular screw head SK1 of the bone screw K1 is partially accommodatable in the first slot L1, being supported on the support face AK1 of the bevel F1.

In the present example embodiment, the slot L1 is annularly closed, wherein the bevel F1 extends peripherally on the top side O of the slot L1.

As is apparent in FIG. 1c in particular, a single thread projection S1 is formed on the first longitudinal bar LL1 underneath the bevel F1 in the slot L1. The thread projection S1 is configured such that it is accommodated in a thread turn GK1 of the bone screw K1.

Underneath the bevel F1 in the first slot L1, a flat surface EF is formed on the second longitudinal bar LL2, which flat surface extends substantially in parallel with a depth extent T of the first slot L1. The flat surface EF extends between the top side O and the bottom side U. Therefore, a geometry for accommodating a thread turn of the bone screw K1 is not provided on the second longitudinal bar LL2.

A first and a second marker M1, M2 are provided along the longitudinal bars LL1, LL2 on the top side O, which indicate target positions of the bone screw K1 for the surgeon.

The second slot L2 is designed similarly to the first slot L1.

Due to this example design, the bone screws K1, K3, K4 in the first and the second slots L1, L2 are bringable into engagement not only at different positions, but also with a variable inclination with respect to the depth extent T of the first and the second slots L1, L2 to be anchored to the bone.

FIG. 1d shows a slot L1′ in a modified example design, in that slot L1′ has a first section A1 and a second section A2, in each of which the longitudinal bars LL1, LL2 extend in parallel with one another, wherein the first section A1 and the second section A2 extend at an angle relative to one another, the reference character K indicating a bending point.

Otherwise, the modified first slot L1′ is designed similarly to the first slot L1.

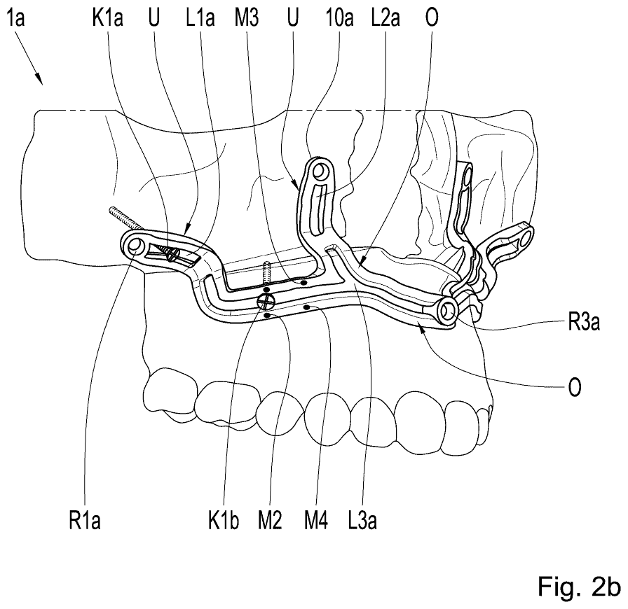

FIG. 2a and FIG. 2b are schematic views of an osteosynthesis implant system according to a second example embodiment, specifically FIG. 2a showing a perspective view of a planned connection state between a skull bone SK and an upper jaw bone OK, and FIG. 2b showing a perspective view, in sections, of a connection state between a skull bone SK and an upper jaw bone OK with two bone screws inserted in slots.

With reference to FIG. 2a, reference character 1a generally designates the osteosynthesis implant system according to the second example embodiment, which is shown in an implanted state in which it is used to stabilize a gap FP between an upper jaw bone OK and a skull bone SK. For the sake of clarity, the bone screws are not shown in FIG. 2a.

The osteosynthesis implant system 1a includes, in addition to the bone screws (not shown), which are designed as in the first example embodiment, an osteosynthesis plate 10a having a bottom side U that is to rest against the bone and a top side O facing away from the bone.

The osteosynthesis plate 10a has a first through sixth slot L1a through L6a, which are designed similarly to the slots L1, L2 and L1′, L2 of the first example embodiment. A first through fourth marker M1 through M4 are provided along the longitudinal bars of the slot L3a on the top side O, which indicate target positions of the bone screw for the surgeon.

The osteosynthesis plate 10a also has a first through fifth round hole R1a through R5a in which the bone screws (not shown) are bringable into engagement at a fixedly defined position to be anchored to the skull bone SK and to the upper jaw bone OK.

Structurally, the osteosynthesis plate 10a includes a curvilinear region B1, which has a central round hole R3a. The slot L3a and the slot L4a are provided adjacent to the central round hole R3a on both sides and cover the entire extent of the curvilinear region B1.

Proceeding from the curvilinear region B1, the osteosynthesis plate 10a includes a first through fourth finger region BF1 through BF4 which have the slot L1a, L2a, L5a, L6a, respectively, to which the round hole R1a, R2a, R4a, and R5a, respectively, adjoins.

A first divider ST1 is provided between the slot L1a and the slot L3a, a second divider ST2 is provided between the slot L2a and the slot L3a, a third divider ST3 is provided between the slot L4a and the slot L5a, and a fourth divider ST4 is provided between the slot L4a and the slot L6a.

This design enables slots L1a through L6a to be provided over a large surface and offers the surgeon the greatest possible flexibility with regard to inserting the bone screws, as is shown in FIG. 2b on the basis of the bone screw K1a in the slot L1a and on the basis of the bone screw K1b in the slot L3a.

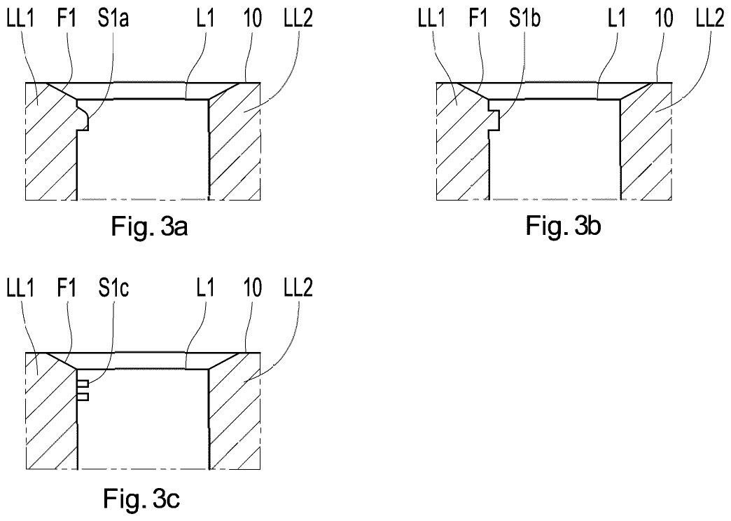

FIG. 3a through FIG. 3c are schematic sectional representations of example variants of the thread projection in further example embodiments of the present invention.

As is apparent in FIG. 3a, a single thread projection S1a is formed on the first longitudinal bar LL1 underneath the bevel F1 in the first slot L1, which thread projection has a nose-shaped monoprojection shape.

As is apparent in FIG. 3b, a single thread projection S1b is formed on the first longitudinal bar LL1 underneath the bevel F1 in the first slot L1, which thread projection has a rectangular monoprojection shape.

As is apparent in FIG. 3c in particular, two thread projections S1c are formed on the first longitudinal bar LL1 underneath the bevel F1 in the first slot L1.

A polyprojection shape means, in particular, that the polyprojection as well as the monoprojection engage in the thread turn of the bone screw only at one point.

Although the present invention has been described above on the basis of preferred exemplary embodiments, it is not limited thereto and instead can be modified in a variety of ways. In particular, the geometry of the osteosynthesis plate, of the bone screw, and, in particular, of the slots, can be varied in a variety of ways.

Modifications and variations can be made to the embodiments illustrated or described herein without departing from the scope and spirit of the invention as set forth in the appended claims. In the claims, reference characters corresponding to elements recited in the detailed description and the drawings may be recited. Such reference characters are enclosed within parentheses and are provided as an aid for reference to example embodiments described in the detailed description and the drawings. Such reference characters are provided for convenience only and have no effect on the scope of the claims. In particular, such reference characters are not intended to limit the claims to the particular example embodiments described in the detailed description and the drawings.

### LIST OF REFERENCE CHARACTERS

1, 1a osteosynthesis implant system
FP gap
OK upper jaw bone
SK skull bone
K1-K6, K1a, K1b bone screws
10, 10′, 10a osteosynthesis plate
U bottom side
O top side
L1, L2, L1′, L1a-L6a slot
LL1-LL4 longitudinal bars
R1-R3, R1a-R5a round hole
F1 bevel
SK1 screw head
AK1 support face
S1, S1a, S1b, S1c thread projection
M1, M2, M3, M4 marker
T depth extent
P arrow
GK1 thread turn
EF flat surface
A1, A2 section
K bending point
B1 curvilinear region
BF1-BF4 finger region
ST1-ST4 divider

1-15. (canceled)

16. An osteosynthesis implant system (1; 1a) comprising:
a bone screw (K1; K2, K3; K1a, K1b) having a screw head (SK1) and a screw shank; and
an osteosynthesis plate (10; 10′; 10a) having a bottom side (U) configured for resting against a bone (SK, OK) and a top side (O) facing away from the bone (SK, OK), the osteosynthesis plate (10; 10′; 10a) comprising at least one slot (L1, L2; L1′, L2; L1a-L6a) through which the bone screw (K1; K2, K3; K1a, K1b) is bringable into engagement at different positions to be anchored to the bone (SK, OK); the osteosynthesis plate (10; 10′; 10a) further comprising a first longitudinal bar (LL1) and a second longitudinal bar (LL2), the second longitudinal bar (LL2) arranged opposite and parallel with the first longitudinal bar (LL1), the first and second longitudinal bars (LL1, LL2) extending along an extension direction of the osteosynthesis plate (10; 10′; 10a) and each having, at the top side (O), a bevel (F1) that is directed into the slot (L1, L2; L1′, L2; L1a-L6a) and is configured such that the screw head (SK1) is at least partially accommodatable in the slot (L1, L2; L1′, L2; L1a-L6a) in a supported manner such that a contact surface for the screw head (SK1) is provided on the surface formed by the bevel (F1),
wherein a thread projection (S1) for accommodating a thread turn (GK1) of the screw shank is formed on the first longitudinal bar (LL1) underneath the bevel (F1), and a flat surface (EF) is formed on the second longitudinal bar (LL2) underneath the bevel (F1), the flat surface extending substantially in parallel with a depth extent (T) of the slot (L1, L2; L1′, L2; L1a-L6a).

17. The osteosynthesis implant system (1; 1a) of claim 16, wherein the second longitudinal bar (LL2) does not have a geometry to accommodate the thread turn (GK1).

18. The osteosynthesis implant system (1; 1a) of claim 16, wherein the flat surface (EF) extends between the top side (O) and the bottom side (U).

19. The osteosynthesis implant system (1; 1a) of claim 16, wherein the slot (L1, L2; L1′, L2; L1a-L6a) is annularly closed and the bevel (F1) extends peripherally on the top side (O) of the slot (L1, L2; L1′, L2; L1a-L6a).

20. The osteosynthesis implant system (1; 1a) of claim 16, wherein the bone screw (K1; K2, K3; K1a, K1b) is bringable into engagement in the slot (L1, L2; L1′, L2; L1a-L6a) at the different positions with a variable inclination with respect to the depth extent (T) of the slot (L1, L2; L1′, L2; L1a-L6a) to be anchored to the bone (SK, OK).

21. The osteosynthesis implant system (1; 1a) of claim 16, wherein the screw head (SK1) of the bone screw (K1; K2, K3; K1a, K1b) is lenticular.

22. The osteosynthesis implant system (1; 1a) of claim 16, wherein the thread projection (S1; S1a; S1b) is formed by a single projection.

23. The osteosynthesis implant system (1; 1a) of claim 16, wherein the thread projection (S1c) is formed by at least two consecutively arranged individual projections.

24. The osteosynthesis implant system (1; 1a) of claim 16, wherein the thread projection (S1; S1a; S1b) is arranged at half the height between a lower edge of the bevel (F1) and an edge of the first longitudinal bar (LL1) to the bottom side (U).

25. The osteosynthesis implant system (1; 1a) of claim 16, wherein the thread projection (S1; S1a; S1b) has a non-symmetrical profile.

26. The osteosynthesis implant system (1; 1a) of claim 16, wherein the osteosynthesis plate (10; 10′; 10a) comprises at least one round hole (R1, R2, R3; R1a-R5a) through which the additional bone screw (K2; K5, K6) is bringable into engagement at a fixedly defined position to be anchored to the bone (SK, OK).

27. The osteosynthesis implant system (1; 1a) of claim 16, wherein the osteosynthesis plate (10; 10′; 10a) comprises a first plurality of slots (L1, L2; L1′, L2;
L1a-L6a) and a second plurality of round holes (R1, R2, R3; R1a-R5a), wherein at least one of the round holes (R1, R2, R3; R1a-R5a) adjoins one of the slots (L1, L2; L1a-L6a).

28. The osteosynthesis implant system (1; 1a) of claim 16, wherein:
the slot (L1′) comprises a first section (A1) and a second section (A2);
in each of the first and second sections (A1, A2) the first and second longitudinal bars (LL1, LL2) extend in parallel;
the first section (A1) and the second section (A2) extend at an angle relative to each other.

29. The osteosynthesis implant system (1; 1a) of claim 16, wherein one or more marker(s) (M1, M2; M1-M4)) is provided along the longitudinal bars (LL1, LL2) on the top side (O) to indicate target positions of the bone screw (K1; K2, K3; K1a, K1b) in the slot (L1, L2; L1a-L6a).

20. The osteosynthesis implant system (1; 1a) of claim 16, wherein the osteosynthesis plate (10; 10′; 10a) is a maxilla repositioning implant for orienting a maxilla displacing portion (OK) relative to a remaining maxilla portion (SK) fixed to the skull.
